(19) United States
(12) Patent Application Publication
(10) Pub. No.: US 2025/0098966 A1
(43) Pub. Date: Mar. 27, 2025

(54) AUTOMATIC CONTROL OF A MEASUREMENT TIME OF AN IMPLANTABLE DEVICE

(71) Applicant: BIOTRONIK SE & Co. KG, Berlin (DE)

(72) Inventors: Christian MOSS, Falkensee (DE); Andreas ARNDT, Berlin (DE); Olaf SKERL, Bad Doberan (DE); Franziska WEGERICH, Berlin (DE)

(73) Assignee: BIOTRONIK SE & Co. KG, Berlin (DE)

(21) Appl. No.: 18/728,226

(22) PCT Filed: Jan. 23, 2023
(86) PCT No.: PCT/EP2023/051502

§ 371 Date: Jul. 11, 2024

(30) Foreign Application Priority Data
Jan. 25, 2022 (EP) 22153074.4

(51) Int. Cl. A61B 5/0215; A61B 5/0205

(52) U.S. Cl. CPC A61B 5/0215; A61B 5/02055

(57) ABSTRACT

An implantable device comprising means for deriving a circadian rhythm associated with a physiological parameter. The implantable device further comprises means for determining a blood pressure, based at least in part on the circadian rhythm. Further aspects relate to a method carried out by such a device and a computer program.

### CROSS-REFERENCE TO RELATED APPLICATIONS

This application is the United States National Phase under 35 U.S.C. § 371 of PCT International Patent Application No. PCT/EP2023/051502, filed on Jan. 23, 2023, which claims the benefit of European Patent Application No. 22153074.4, filed on Jan. 25, 2022, the disclosures of which are hereby incorporated by reference herein in their entireties.

### TECHNICAL FIELD

The present invention generally relates to an implantable device and a method and a computer program for operating such device, particularly to determine a blood pressure based on a derived circadian rhythm of a physiological parameter.

### BACKGROUND

The implantable device is a device which may be implanted into a patient and may be used for monitoring various parameters and/or for actively facilitating therapies (e.g., via electrical stimulations by the device) from within the patient. Common implantable devices may comprise a sensor for sensing a physiological parameter (e.g., a blood pressure, a heart rate, a respiratory rate, a nerve activity, etc.). The physiological parameter may not necessarily be measured continuously over time. It may be adequate for some applications to measure the physiological parameter at a certain (e.g., discrete) measurement time. For example, it may be necessary to regularly measure a physiological parameter for monitoring the health status of a patient (e.g., via daily checks). To that regard, it may be beneficial to set the measurement time in a resting phase of the patient to enable comparable measurement results (since the patient's activity may influence the intrinsic properties of a physiological parameter). This approach may enable measurements under reproducible conditions which may lead to a reliable assessment of the physiological parameter and thus the patient's health status.

Known approaches may rely on a manually programmed measurement time in which the patient having the implant is assumed to be in the resting phase (e.g., a sleeping phase). For example, the measurement time may be set by an external device in communication with the implantable device. Known approaches may thus be based on fixedly setting a constant measurement time via a communication link (e.g., the measurement time may be set to be constantly at 4 am when the patient is assumed to be sleeping). However, this approach may suffer from the fact that the measurement time must be manually set (e.g., by a specially trained medical/technical personnel) to fit the patient specific resting phase which may not be constant over the patient's lifetime.

Hence, the currently known approaches do not lead to an optimum assessment of physiological parameters of implantable devices.

The present disclosure is directed toward overcoming one or more of the above-mentioned problems, though not necessarily limited to embodiments that do.

### SUMMARY

The aspects described herein address the above need at least in part.

A first aspect relates to an implantable device which may comprise means for deriving a circadian rhythm associated with a physiological parameter. The implantable device may further comprise means for determining a blood pressure, based at least in part on the circadian rhythm.

The underlying idea of the present invention may be based on the mechanism that various physiological parameters of an organism undergo a circadian rhythm. The circadian rhythm may result from the fact that various organisms have adapted to the changing (but more or less repeating) conditions over a day due to the rotation of the earth around its axis every 24 hours. Due to biological reasons the circadian rhythm of some physiological parameters may be approximately 24 hours, wherein for other physiological parameters the circadian rhythm may be exactly 24 hours. The circadian rhythm may be considered a process that regulates at least one characteristic (e.g., a value, a frequency, etc.) of a physiological parameter of an organism wherein the characteristic may repeat itself (approximately) every 24 hours. To that regard, the period of (approximately) 24 hours may be referred to as one cycle of the circadian rhythm. In an example, the characteristics of a physiological parameter (regulated by the circadian rhythm) may be defined by one local maximum and/or one local minimum in a time period of (approximately) 24 hours. In that example, the one local maximum and/or one local minimum may occur at (approximately) the same time, respectively, for every day. However, the circadian rhythm may change due to external influences (e.g., a local change of the organism may induce a shift in the prior day-night cycle, a change in light accessibility, etc.) which may impact the time characteristics of the circadian rhythm. As a response to the changed circumstances, the circadian rhythm of the organism may realign (e.g., shift in time) and continue its characteristic oscillation (approximately) every 24 hours.

Common approaches of implantable devices do not consider the dynamics of the circadian rhythm of an organism. For example, a patient may have an implantable device for determining the patient's blood pressure. Active phases of the patient (e.g., physical activities) may extrinsically alter various physiological parameters (e.g., the blood pressure) and should thus be avoided when monitoring the blood pressure. Therefore, it may be necessary to determine the blood pressure at a specific body phase (e.g., a resting phase) of the patient to ensure reproducible measurement conditions. For example, the specific body phase may coincide with a reasonably stable physiological state of the patient (e.g., a resting phase, a certain sleeping phase in the resting phase). The blood pressure measurement may thus be implemented in the specific body phase to ensure a reliable blood pressure tracking since extrinsic influence are minimized. However, the specific body phase may not be constant over time due to the dynamics of the circadian rhythm which may be influenced by the patient's actions, behavior, age and/or external impacts. Thus, a change in the circadian rhythm may affect the time the specific body phase takes place.

Known approaches may suffer from the fact that they use a static approach. For example, known implantable devices may use a fixedly programmed measurement time for determining/measuring the blood pressure wherein the measurement time is guessed to be in the resting phase of the patient (e.g., 4 am in the morning). Initially the measurement time may have to be fixedly programmed after production and/or implantation of the implantable device for technical reasons. However, the resting phase of the patient may not be constant over the patient's life (e.g., due to daylight-savings time and/or, e.g., the patient may: travel to a different time zone, change work shifts, switch his/her resting phase during vacation, etc.). To that regard, common approaches may rely on a manual readjustment of the fixedly programmed measurement time in the implant to guarantee that it coincides with the resting phase. Hence, known implantable device may require a complex bidirectional communication unit in the implant for said purpose which may increase system complexity and increase energy consumption. In addition, this approach may require manual patient interaction. For example, the patient may have to reach out to a dedicated user (e.g., medical/technical personnel) who is specifically trained to operate an external programming unit for manually readjusting the settings of the implantable device since the patient may not have access (e.g., for safety reasons) to the communication interface of the implant. This may not be considered beneficial since it requires an active awareness of the outlined problem by the patient and his active interaction with the dedicated user who may not be easy to reach (e.g., when traveling into a country with a different time zone). In general, the known approaches may not achieve an automatic adjustment of an optimum measurement time.

The concept of the present invention avoids the known drawbacks since it may consider the circadian rhythm dynamics in an organism for determining the blood pressure under reproducible measurement conditions. Specifically, the blood pressure may be determined (e.g., a measurement may be triggered) at a measurement time or measurement time interval that is based on the circadian rhythm. Hence, the measurement may consistently be made at an optimum measurement time (e.g., a resting phase of the patient) over time.

By deriving the circadian rhythm associated with a physiological parameter a characteristic (e.g., a characteristic moment of time) in the circadian rhythm may be correlated with the specific body phase. The specific body phase may coincide with a reasonably stable physiological state of the patient as outlined herein. A body phase may be an activity phase (e.g., a resting phase, an active phase of a human) which may correlate with the circadian rhythm of the physiological parameter. Notably, the body phases may also undergo a circadian rhythm. The inventive concept may thus enable determining the specific body phase by deriving the circadian rhythm. In addition, this concept allows setting a measurement time for determining the blood pressure in the specific body phase based on the derived circadian rhythm without external or manual intervention. This may enable a (fully) automated autarkic approach to determine the blood pressure which may minimize external interactions with the implant but at the same time provides consistent and comparable blood pressure values over time. For example, a setting of a measurement time may not be necessary during production and/or implantation since the implant may automatically determine an optimum time for determining the blood pressure based on the circadian rhythm. Further, the present invention may be highly beneficial when the circadian rhythm (and thus the specific body phase) of the patient changes in time. Since the circadian rhythm (and thus the specific body phase) may be regularly monitored by the implantable device, the time for determining the blood pressure may be realigned with the current time of the specific body phase. Therefore, the present invention may enable to continuously determine the blood pressure under reproducible conditions (i.e., in the same (stable) specific body phase), which may lead to a better health tracking. Resulting from the automatic approach less communication requirements (e.g., less hardware requirements) may be necessary which may lead to a reduced system complexity, a reduced energy consumption, an increased device longevity, as well as a smaller implant which may cause less medical interference for the patient.

The means for deriving a circadian rhythm may comprise a computing unit (e.g., a microcontroller, an ASIC, a microprocessor, etc.) for implementing the necessary processing steps for deriving the circadian rhythm. The means for deriving may be coupled to an interface which may input data associated with the physiological parameter to the means for deriving. The data input may comprise a data set (e.g., values) of the physiological parameter over a certain time period, for example. The means for deriving may be configured to implement various types of signal processing to the data input for deriving the circadian rhythm (e.g., filtering, feature extraction, amplitude sampling, computational checks etc.). The signal processing may be carried out by hardware (e.g., by a signal processing unit, a microcontroller, an ASIC, a microprocessor, etc.) and/or software comprised in the implantable device.

In an example, the means for deriving may be configured to analyze if a certain characteristic of the physiological parameter may repeat itself (approximately) every 24 hours. This information may be used to derive the circadian rhythm associated with the physiological parameter. In one example, the derived circadian rhythm may be based on analyzing one cycle of the circadian rhythm (i.e., 24 hours). The means for deriving may further be configured to base the analysis of the circadian rhythm on an (arbitrarily) chosen cycle start to enable a reference point for further investigations. The cycle start may be based on the initial value of a physiological parameter available for analysis. In another example the derived circadian rhythm may be based on analyzing a plurality of cycles (i.e., a plurality of days, a plurality of 24-hour time windows, analyzing the physiological parameter over a time greater than 24 hours). When analyzing a plurality of cycles, the means for deriving may be configured to average the respective results of each one of the plurality of cycles into a global circadian rhythm. This may increase the accuracy, result in a higher confidence level of the derived circadian rhythm and/or may reduce measurement error. For example, the implant may analyze two consecutive cycles of the physiological parameters. The local minimum of the first cycle may appear 7 hours after the first cycle start. The local minimum of the second cycle may appear 6.5 hours after the second cycle start. Hence, in that example the device may average the results (e.g., (7 hours+6.5 hours)/2=6.75 hours)) to derive at the most probable time location of the local minimum after a cycle start (e.g., 6.75 hours after cycle start).

To illustrate an example, the means for deriving may be configured to determine if one local maximum and/or minimum of the physiological parameter is present over 24 hours. It may be optionally further configured to check if the local maximum and/or minimum repeats (approximately) every 24 hours. The means for deriving may further be configured to determine if the physiological parameter represents a periodical signal type over time (e.g., if it fits a sine, a cosine, a periodical rectangular function, etc.). In particular, the periodical signal type may be analyzed regarding a period of a circadian cycle (i.e., approximately 24 hours).

In an example, the means for deriving may be configured to check if the physiological parameter undergoes a (significant) circadian rhythm and/or what type of circadian rhythm is present (e.g., what signal type, signal shape may be present). This may be used to further analyze the circadian rhythm and/or derive the circadian rhythm. In an example, the derived circadian rhythm may correlate with the sleep-wake cycle of the patient having the implant.

In another example, the implantable device may be configured to consider a plurality of physiological parameters, wherein for each physiological parameter a respective circadian rhythm is derived. In another example, the means for deriving may be configured to derive an overall circadian rhythm which may be based on the plurality of physiological parameters (e.g., the implantable device may average the respective circadian rhythms of each one of the plurality of physiological parameters into a global circadian rhythm which may have a higher confidence level and lower measurement error).

In an example, the implantable device may couple the information of the derived circadian rhythm (e.g., its characteristics, the specific body phase, etc.) to the means for determining the blood pressure. In another example, the implantable device may couple a trigger to the means for determining which may trigger a measurement (or an active determination) of the blood pressure by the means for determining.

In an example, the means for determining may be a first system for sensing and/or determining the blood pressure. For example, the first system may comprise a sensor unit, a sensor and/or sensing element for sensing the blood pressure signal. The blood pressure may be determined by the first system from the sensed blood pressure signal by applying signal processing to the sensed signal. The signal processing may be carried out by hardware (e.g., by a signal processing unit, a microcontroller, a microprocessor, an ASIC, an embedded system, etc.) and/or software comprised in the first system and/or the sensor device.

In an example, the implantable device may further comprise means for acquiring the physiological parameter. For example, means for acquiring may be a second system for sensing and/or determining the physiological parameter. For example, the second system may comprise a sensor unit, a sensor and/or sensing element for sensing the physiological parameter (e.g., from within the patient). The physiological parameter may be determined by the second system from the sensed physiological parameter signal by applying signal processing to the sensed signal. The signal processing may be carried out by hardware (e.g., by an analog-to-digital converter, a signal processing unit, a microcontroller, a microprocessor, an ASIC, an embedded system, etc.) and/or software comprised in the second system and/or the implantable device. The means for acquiring may thus provide an acquired physiological parameter signal for further processing.

In an example, the means for acquiring may be coupled to the interface to input the acquired physiological parameter to the means for deriving. For example, the means for deriving may be configured to derive the circadian rhythm based at least in part on the acquired physiological parameter. In another example, the means for acquiring may be configured to receive the physiological parameter externally (e.g., from another implantable device, another implantable sensor, an external device residing outside of the patient). For example, the means for acquiring may comprise a communication unit for receiving the physiological parameter.

In an example, the means for deriving and/or the means for determining and/or the means for acquiring may share one or more elements with each other. For example, the means for determining may share hardware (e.g., a power supply, a microcontroller, a storage unit etc.) and/or software with the means for determining and/or means for acquiring. In another example, one main unit (i.e., one local entity) may comprise some elements or all elements of the means for deriving and/or the means for determining and/or the means for acquiring (e.g., inside one casing of the main unit).

In an example, the means for acquiring may be configured to acquire the physiological parameter in predetermined time intervals. Since the circadian rhythm may be considered an oscillation with a period of (approximately) 24 hours it may not be necessary to continuously acquire the physiological parameter for gaining sufficient information on the characteristics of the circadian rhythm. For example, it may suffice to acquire (e.g., sample) the physiological parameter in (discrete) predetermined intervals such that the characteristic of the circadian rhythm may still be derived. Hence, the means for acquiring may repeatedly acquire the physiological parameter after a predetermined time interval Δt has passed (e.g., the physiological parameter may be acquired every minute, every 30 minutes, every hour, every 2 hours, every 4 hours, every 6 hours, etc.). This approach may reduce energy consumption due to the reduction in computing/sensing complexity resulting from the reduced sampling of the physiological parameter without losing information on the derived circadian rhythm characteristics. In an example, the device may be configured for automatic and/or manual adjustment of the values of the predetermined time intervals. For example, the device may be configured to execute a program which may adapt the predetermined time intervals. This may be based on prior derivations of the cardiac rhythm (e.g., a first cycle may be sampled with a first Δt, and a second cycle may be sampled with a second Δt). It may also be conceivable, that the implantable device may communicate with an external device wherein a user may input the dimensions of the predetermined time intervals.

In an example, the means for deriving may be configured to derive at least one circadian rhythm parameter based at least in part on the circadian rhythm. For example, with the circadian rhythm being an oscillation signal with a period of (approximately) 24 hours various circadian rhythm parameter associated with said oscillation signal may be derived. In an example, the at least one circadian rhythm parameter may be based on the acquired physiological parameter signal in general. For example, the at least one circadian rhythm parameter may comprise at least one of the following: a mesor, a signal offset, an amplitude, a period, a frequency, a minimum value, a local minimum value, a maximum value, a local maximum value, a shape, a signal phase, an average value, an effective value, etc. The at least one circadian rhythm parameter may be based on a signal transformation of the acquired physiological parameter signal (e.g., a Fourier transformation, a Laplace transformation, etc.). The at least one circadian rhythm parameter may be based on any signal processing applied to the physiological parameter signal (e.g., filtering, derivation, integration, etc.). In another example, a temporal position in the cycle associated with the at least one circadian rhythm parameter may also be determined. For example, certain circadian rhythm parameters may have a temporal position with reference to a chosen cycle start/end (e.g., the local minimum/maximum value, a certain signal phase may be present after a certain time has passed after the beginning of a cycle).

In an example, the means for determining may be configured to determine the blood pressure based at least in part on the at least one circadian rhythm parameter. For example, the determining of the blood pressure may thus be synchronized with the at least one circadian rhythm parameter. For example, the circadian rhythm parameter may be used to determine a reproduceable measurement time for determining the blood pressure. In an example, the blood pressure may be determined based at least in part on the temporal position of the at least one circadian rhythm parameter. The means for deriving may have derived that a local minimum of the physiological parameter may take place 6 hours after the cycle start. Hence, the means for determining may determine the blood pressure 6 hours after the cycle start (which may be communicated by the means for deriving to the means for determining). To illustrate another example, the means for determining may be configured to determine the blood pressure if the means for deriving has derived that a local minimum of the physiological parameter is currently present.

In an example, the at least one circadian rhythm parameter may comprise a characteristic interval of the circadian rhythm. For example, a characteristic interval may be associated with a certain time frame in the circadian rhythm. In another example, the characteristic interval may be defined by a certain value range of the physiological parameter. The characteristic interval of the circadian rhythm may be medically relevant and may not necessarily be defined by a mere mathematical characteristic. The characteristic interval may be an interval spanning around the local minimum and/or the local maximum in a cycle of the circadian rhythm (i.e., the physiological parameter). For example, the local maximum may be positioned at 6 hours after a cycle start, wherein one characteristic interval may span between 5 hours to 7 hours after the cycle start. In that example, the physiological parameter value at 5 hours and 7 hours after cycle start may not be less than 30% of the maximum amplitude of the physiological parameter signal.

In an example, the characteristic interval may comprise a bathyphase of the circadian rhythm and/or an acrophase of the circadian rhythm. For example, the bathyphase may be considered a phase in which a human may have his/her minimum activity (e.g., a resting phase). In addition, the bathyphase may be the characteristic interval in which the body temperature of a human is at its minimum. In an example, the deriving of the bathyphase may be based on acquiring the physiological parameter over a prolonged period of time (e.g., multiple days) to achieve a higher accuracy. Notably, the bathyphase may be a time interval in a cycle of the circadian rhythm during which the cycle is low (e.g., a lower part of a sine wave fitted to the acquired physiological parameter signal). The means for deriving may be configured to determine a bathyphase center time in a cycle (e.g., with reference to the chosen cycle start) which may represent the center of the bathyphase. It may further be configured to determine the bathyphase start time, and the bathyphase end time in a cycle (e.g., with reference to the chosen cycle start) which may be based on a medical characteristic.

The acrophase may be considered a phase in which a human may have his/her maximum activity (e.g., an active phase). In addition, the acrophase may the characteristic interval in which the body temperature of a human is at its maximum. The external (e.g., changing) conditions in the acrophase may be unpredictable. Hence, the physiological parameters (i.e., the blood pressure) may be highly volatile from day to day due to the patient's varying activities (e.g., athletic activities, various changes in body position during a workday, etc.). In an example, the deriving of the acrophase may be based on acquiring the physiological parameter over a prolonged period of time (e.g., multiple days) to achieve a higher accuracy. Notably, the acrophase may be a time interval in a cycle of the circadian rhythm during which the cycle peaks (e.g., an upper part of a sine wave fitted to the acquired physiological parameter signal). The means for deriving may be configured to determine an acrophase center time in a cycle (e.g., with reference to the chosen cycle start) which may represent the center of the acrophase. It may further be configured to determine the acrophase start time, and the acrophase end time in a cycle (e.g., with reference to the chosen cycle start) which may be based on a medical characteristic.

In an example, the means for determining may be configured to determine the blood pressure at least in part based on the characteristic interval. For example, the blood pressure may be determined based at least in part on the bathyphase. As outlined herein, the bathyphase may be considered the specific body phase suitable for determining the blood pressure since the physiological conditions in the bathyphase may be relatively stable over time. The implantable device (and/or the means for determining) may thus be configured to determine the blood pressure based at least in part on the bathyphase. For example, the implantable device (and/or the means for determining) may be configured to determine the blood pressure during the bathyphase which may be a suitable measurement time. Notably, the blood pressure may be determined in the bathyphase center time or between the bathyphase start time and the bathyphase end time.

In another example, the blood pressure may be determined based at least in part on the acrophase. However, as outlined herein, the acrophase may not be considered a body phase during which it is preferable to determine the blood pressure since the conditions in the acrophase may be relatively unstable (e.g., patient may be working, exercising, walking, etc.). The acrophase may thus not lead to reproduceable measurement conditions since the physiological parameters (especially the blood pressure) may be highly volatile. Hence, the implantable device (and/or the means for determining) may be configured to determine the blood pressure in a time not associated with the acrophase. For example, if only the acrophase may have been derived the blood pressure may be determined at a shifted time which may correspond to the bathyphase (e.g., at a time shifted by 12 hours regarding the acrophase). Notably, the blood pressure may be determined in a shifted time which may be the acrophase center time shifted by 12 hours.

In an example, the means for determining may be configured to determine the blood pressure based at least in part on a first predetermined timing relative to the characteristic interval. For example, the characteristic interval (e.g., the bathyphase) may be considered a characteristic reference time wherein the subsequent times for determining the blood pressure may be based on a timing (e.g., every 24 hours) relative to the characteristic reference time. This approach takes into account that the circadian rhythm periodically repeats itself every 24 hours. Hence, the subsequent times for determining the blood pressure may not necessarily be based on a further derivation of the circadian rhythm. To illustrate an example, the implantable device may have derived a circadian rhythm and derived the temporal position of the characteristic interval (e.g., a bathyphase center time, a local minimum, etc.) in the cycle of the circadian rhythm optimal for determining a blood pressure. Subsequently, the implantable device may (e.g., temporarily) halt deriving the circadian rhythm since further information may not be needed. In that example, a timer of 24 hours may be started at the temporal position of the characteristic interval. When the timer lapses the blood pressure may be determined wherein the time is started again to run for the next 24 hours which may accordingly trigger a determination of the blood pressure, and so on. To that regard the predetermined timing may not be limited to 24 hours and may comprise any time value (e.g., 12 hours, 48 hours, 72 hours). This inventive approach may thus enable an automated determining of the blood pressure with little computation effort once a (suitable/convenient) temporal position in a cycle for determining the blood pressure has been derived since only a timer is needed for triggering a measurement.

In an example, the means for deriving may be configured to derive the circadian rhythm based at least in part on a second predetermined timing. For example, the circadian rhythm may change over time, as outlined herein. Hence, the inventive approach may consider deriving the circadian rhythm in regular intervals to ensure that the derived characteristics are still present and the determining of the blood pressure may occur in the same temporal position of the cycle. In an example, the deriving of the circadian rhythm may be repeated every week, every two weeks, every month, every two months, every six months, every year. In an example, the second predetermined timing may be greater than the first predetermined timing. This may ensure that the deriving of the circadian rhythm does not occur more frequent than the determining of the blood pressure.

In another example, the circadian rhythm may be readjusted by an external message. For example, the implantable device may be configured to communicate with an external device. The external device may send a synchronization message (e.g., a synchronization time) to the implantable device which may be used to adjust the settings (e.g., of the timer) in the implantable device and/or may trigger the means for deriving to initiate deriving the circadian rhythm. For example, the external device may be adapted by the patient having the implant (e.g., during travel, the patient may communicate to the implant via the simple synchronization message that his/her circadian rhythm may likely change).

In an example, the physiological parameter may comprise at least one of the following: a body temperature, a heart rate, a respiratory rate, a sleeping pattern, a blood pressure, a hormone level, a melatonin level, a plasma level of cortisol, a body activity.

In a presently preferred example, the physiological parameter may comprise the body temperature. The means for determining may thus be configured to determine the blood pressure based at least in part on a derived circadian rhythm associated with an acquired body temperature. In another example, the means for determining may be configured to determine the blood pressure based at least in part on an acquired body temperature. In another preferred example, the means for acquiring may be configured for sensing and/or determining the body temperature.

Accordingly, the implantable device may comprise at least one of the following means for acquiring a physiological parameter: means for acquiring a body temperature, means for acquiring a heart rate, means for acquiring a respiratory rate, means for acquiring a sleeping pattern, means for acquiring a blood pressure, means for acquiring a hormone level, means for acquiring a melatonin level, means for acquiring a plasma level of cortisol, means for acquiring a body activity.

In an example, the implantable device may be configured for implanting into a blood vessel. Hence, the implantable device may be operably configured as a (single) intravascular implant. In an example the implantable device may comprise means for fixing the sensor device to the blood vessel. This may be beneficial for enabling various types of mechanical fixation mechanisms which may be required to ensure a stable fixation to a broad range of blood vessel types. This may ensure a reliable fixation of the implantable device which may lead to a reliable readout of the blood pressure, a reduction of design complexity and/or a position for deriving the circadian rhythm. Notably, the implantable device may be configured for implanting into any artery, elastic artery, distributing artery, vein, arterioles, capillaries, venules, sinusoids of a patient.

In an example, the implantable device may be configured for implanting into a blood vessel, wherein the blood vessel is a pulmonary artery. For example, the position in the region of the pulmonary artery may be a beneficial implant location for the implantable device due to the comparatively wide blood vessel dimensions which may enable a mechanically stable connection. Further, the position in the region of the pulmonary artery may enable an easier implant (or explant) procedure for medical personnel which may result in less medical complications for the patients.

A second aspect relates to a method carried out by an implantable device. The method may comprise deriving a circadian rhythm associated with a physiological parameter. It may further comprise determining a blood pressure, based at least in part on the circadian rhythm.

A third aspect relates to a computer program comprising instructions to perform the method as outlined herein, when the instructions are executed by a processor. In an example the computer program instructions may be stored on a non-transitory medium. For example, the computer program may be stored on the implantable device as described herein, and the latter may comprise means (e.g., a processor) to execute the computer program instructions. The computer program may allow an autarkic, automated implementation of the aspects described herein. Consequently, technical intervention from medical staff and the patient may be minimized.

It is noted that the method steps as described herein may include all aspects described herein, even if not expressly described as method steps but rather with reference to an apparatus (or device). Moreover, the devices as outlined herein may include means for implementing all aspects as outlined herein, even if these may rather be described in the context of method steps.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media include both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, flash memory, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following, the Figures of the present disclosure are listed:

FIG. 1: Schematic representation of an exemplary embodiment of an implantable device.

FIG. 2: Schematic representation of an exemplary embodiment of a circadian rhythm of a physiological parameter.

FIG. 3: Block diagram representing the functional relations of parts of an exemplary embodiment of an implantable device.

### DETAILED DESCRIPTION

Subsequently, presently preferred embodiments will be outlined, primarily with reference to the above Figures. It is noted that further embodiments are certainly possible, and the below explanations are provided by way of example only, without limitation.

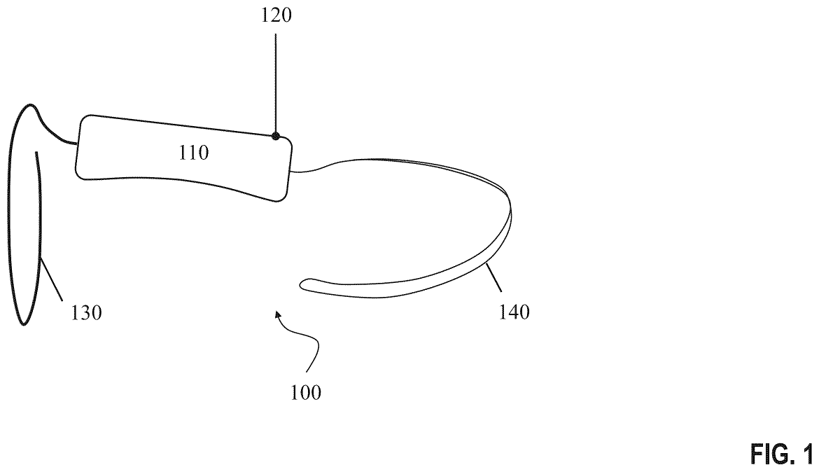

FIG. 1 shows a schematic representation of an exemplary embodiment of an implantable device 100.

The implantable device 100 may comprise a main unit 110. The main unit 110 may encompass various components inside a casing 120, wherein the casing 120 may be of a material chemically durable with respect to an organic and/or aggressive environment. For example, the solid casing 120 may be made of titanium and/or a titanium compound and/or comprise a titanium-based coating to be inert when exposed to an organic/aggressive environment (e.g., blood and/or other organic fluids/tissue, e.g., nerve cells, acid, etc.). The casing of the main unit 110 may thus not significantly chemically react with an organic/aggressive environment which may minimize the influence of the casing onto the organic/aggressive environment and/or may minimize the deteriorating effects of the organic/aggressive environment onto the casing 120 and the components therein.

In an example, the main unit 110 may comprise a membrane (not shown) which may bend under the influence of a pressure of the surrounding environment. The membrane may be of a material which is chemically durable regarding an organic and/or aggressive environment (e.g., titanium and/or a titanium compound and/or comprise a titanium-based coating), as outlined for the casing 120. The casing 120 may comprise an opening, wherein the membrane may be fixedly mounted at its outer edge to the edges of the opening of the casing such that the main unit 110 and its inner components are hermetically sealed from the environment. The membrane may thus freely bend in the area defined by the opening in the casing 120 under the pressure of the environment. This may enable an elastic coupling of the pressure onto the inner parts of the main unit 110 without chemically exposing the inner components of the main unit 110 to the outer environment (e.g., to blood). The membrane may be part of a blood pressure sensor wherein a separate sensor portion may be formed inside the main unit 110 in the area of the membrane. In some examples, one or more parts of the sensor portion may be filled with an oil to couple the pressure to a sensory chip which may sense and/or determine an absolute pressure and/or a relative pressure associated with the pressure. Many types of sensory chips for sensing/determining the absolute and/or relative pressure may be conceivable (e.g., a piezoresistive pressure sensor, a piezoelectric pressure sensor, a capacitive pressure sensor, an electromagnetic pressure sensor, a strain-gauge, an optical pressure sensor, a resonant frequency pressure sensor, a thermal pressure sensor, etc.).

In another example, the main unit 110 may not comprise a membrane. It may be conceivable, that the pressure may be sensed over a pressure sensor which may not require a membrane (e.g., via an optical sensor) wherein the pressure sensor may be comprised by the main unit 110 (e.g., fixedly mounted to the outside of the casing 120). In another example, the pressure sensor may be a separate pressure sensor unit which may be communicatively coupled to the main unit 110 (e.g., over a wire and/or wirelessly). The separate pressure sensor unit may be any type of pressure sensor for sensing/determining the absolute and/or relative pressure configured for an organic/aggressive environment (e.g., a piezoresistive pressure sensor, a piezoelectric pressure sensor, a capacitive pressure sensor, an electromagnetic pressure sensor, a strain-gauge, an optical pressure sensor, a resonant frequency pressure sensor, a thermal pressure sensor, etc.). In such examples, the implantable device may be considered as comprising two separate implantable components that interact with each other.

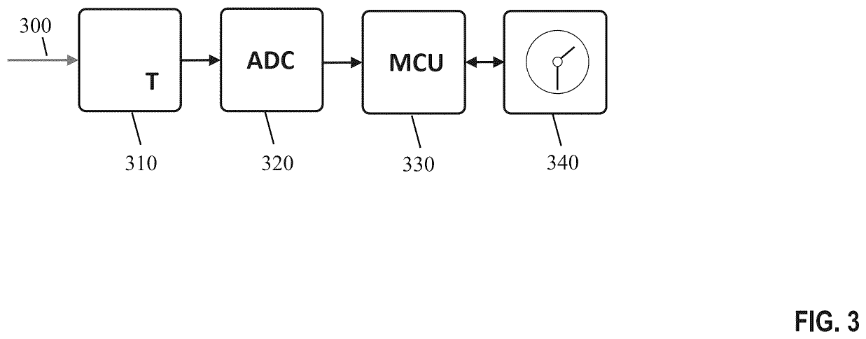

The main unit 110 may comprise further components which are functionally outlined in detail in FIG. 3. For example, the main unit 110 may comprise a sensor unit for sensing a physiological parameter, e.g., a temperature sensor unit 310, an analog-to-digital converter 320, a microcontroller 330 and/or a timer 340. In an example, the temperature sensor unit 310 may be comprised in the main unit 110. In another example, the temperature sensor unit 310 may be a separate implantable sensor unit which may be communicatively coupled to the main unit 110 (e.g., over a wire and/or wirelessly), similarly as outlined with respect to the pressure sensor as outlined above. In such examples, the implantable device may comprise even three separate implantable components that interact with each other.

Preferably, at least one of the pressure sensors and the sensor unit for sensing a physiological parameter may be included in the main unit 110. In some examples, only a pressure sensor may be included and the sensed physiological parameter may be the pressure.

The implantable device 100 may further comprise a first fixation mechanism 130 and a second fixation mechanism 140. The first fixation mechanism 130 and the second fixation mechanism 140 may be made of various materials feasible for mechanically fixing the main unit 110 (and thus the implantable device 100) to a narrow organic geometry (e.g., a blood vessel). For example, the material may be durable regarding the organic/aggressive environment, as outlined herein (e.g., the fixation mechanism 130/140 may be made from titanium, a titanium-compound, e.g., nitinol, comprise a titanium coating, etc.). It may also be conceivable that the material may be made from any other suitable metal, plastic, polymer and/or any combination thereof. The fixation mechanisms 130/140 may further comprise various geometries for mechanically fixing the sensor device 100 to a narrow organic geometry (e.g., a blood vessel). For example, the fixation mechanisms 130/140 may comprise a wire geometry, a strip geometry (e.g., a flat shape, a band shape), tines, a screw shape, a hollow tubular shape. In an example, the fixation mechanisms 130/140 may be based on a stent structure.

In an example, the implantable device 100 may be specifically configured for implanting into a pulmonary artery of a human. The implantable device 100 may be implanted such that the fixation mechanism 130 and the fixation mechanism 140 clamp the implantable device 100 to the pulmonary artery without hindering the flow of blood through the pulmonary artery. For example, the first fixation mechanism 130 may comprise a circular or a spiral shape which mechanically attaches to the inner wall of the pulmonary artery such that blood flow may not be blocked by a disturbance in the center of the cross section of the blood vessel. The second fixation mechanism 140 may be configured to extend over a longer segment in the pulmonary artery than the first fixation mechanism 130 which may lead to an improved fixation of the implantable device to the pulmonary artery.

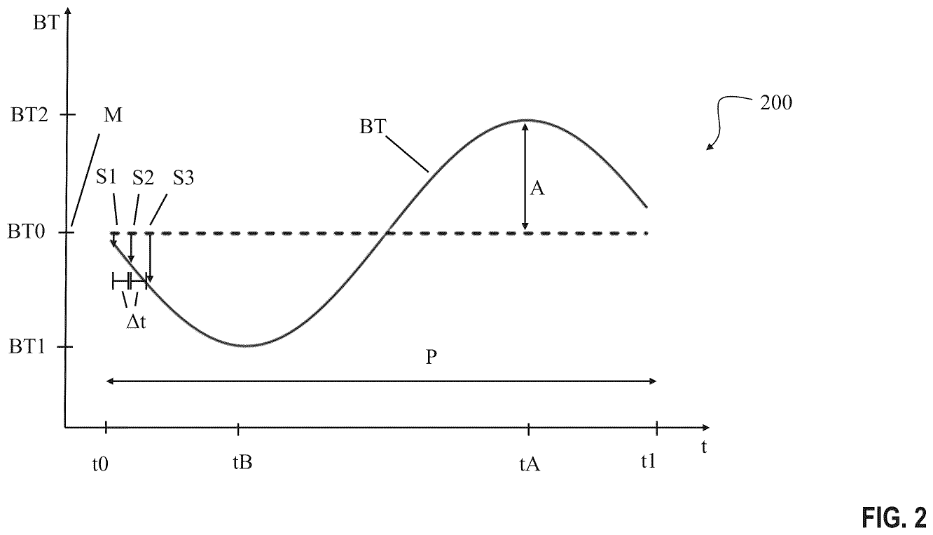

FIG. 2 shows a schematic representation of an exemplary embodiment of deriving a circadian rhythm 200 of a physiological parameter according to the present invention. In this example, the physiological parameter may be the body temperature BT of the patient which is plotted over a time t in FIG. 2. The circadian rhythm of the physiological parameter may be derived by the implantable device 100. As can be seen in FIG. 2 the body temperature BT may undergo a full oscillation over time between an initial time to and a further time t1. The period P (i.e., the difference between t1 and to in FIG. 2) of the body temperature BT may coincide with a circadian cycle of approximately 24 hours. For example, the initial time to and the further time t1 may span a day wherein to may be at 0 am and t1 may be at 12 pm. Due to the circadian rhythm, the curve progression of the body temperature BT may periodically repeat itself (approximately) every 24 hours (i.e., every day). Deriving a circadian rhythm may be understood to require at least the identification of a local maximum (tB) or a local minimum (tA) of the physiological parameter in the course of the day.

Usually, a physiological parameter (e.g., a body temperature BT) that undergoes a circadian rhythm may oscillate around a mesor M value and may show a characteristic amplitude A with reference the mesor M value. In the example of FIG. 2 the mesor M value may be a characteristic body temperature BT0 which may be BT0=37° C. for a patient. In that example, the amplitude may be A=0.5°. In some cases, a circadian cycle of a circadian rhythm may (approximately) match a sine/cosine curve as illustrated in FIG. 2. The characteristic amplitude A is shown for a local maximum of BT2 at a time tA wherein BT2 may be 37.5° C. (resulting from BT2=BT0+A). In accordance, the sine curve of the body temperature BT defines a local minimum of BT1 at a time tB wherein BT1 may be 36.5° C. (resulting from BT1=BT0−A). In the illustrated example, the local minimum of BT1 may be in the bathyphase (wherein tB may be at 6 am) and the local maximum of BT2 may be in the acrophase (wherein tA may be at 6 pm) as outlined herein. The bathyphase of the physiological parameter (e.g., the body temperature BT) may correlate with the bathyphase of the physical activity of the patient. Hence, the minimum body temperature BT1 at the time tB may correlate with a minimum of physical activity of the patient at the time tB. To that regard, the bathyphase/acrophase may not be a distinct point in time but a time interval spanning around the local minimum/maximum of a circadian cycle. As outlined herein, it may be of high interest to determine the blood pressure in the bathyphase by the implantable device 100.

Hence, the implantable device 100 may initially acquire the physiological parameter (e.g., the body temperature BT) to gain information on the circadian rhythm and/or circadian cycle. For example, the body temperature BT in FIG. 2 may be acquired by the sensor unit 310 outlined in FIG. 3. As can been seen in the example of FIG. 2, it may not be necessary to continuously sense the physiological parameter (e.g., the body temperature BT) to derive circadian rhythm characteristics (e.g., the bathyphase). This may result from the comparatively long period P of 24 hours wherein changes in characteristics do not (from a relative perspective) occur abruptly (e.g., characteristic changes do not occur over seconds, in an example characteristic changes may rather occur in the range of an hour). As illustrated in FIG. 2, it may thus suffice to acquire the body temperature BT in predetermined time intervals Δt to track the curve of the circadian cycle. Hence, this approach may be referred to as a sampling of the body temperature BT curve. For exemplary purposes, three sample events S1, S2, S3 are illustrated which may be spaced apart by the predetermined time interval Δt. At every sample event (S1, S2, S3), the body temperature BT may be acquired at a discrete time. Hence, with a plurality of sample events over the course of a day (i.e., a circadian cycle) the curve of the body temperature BT may be acquired in discrete (digital) values. For example, the dotted lines along the mesor M value and BT0 value may be considered further sample events used for acquiring the body temperature BT curve as outlined herein. The predetermined time interval Δt may be set as a constant (e.g., 30 minutes, 1 hour, 2 hours, 4 hours, etc.). It may also be conceivable that the sampling interval (i.e., the predetermined time interval Δt) may be set to vary when acquiring the circadian cycle of a physiological parameter over the course of 24 hours. For example, the sampling interval may be reduced if the parameter changes from one interval to the other reduce, which may indicate that the parameter is close to a local minimum or maximum (which may be indicative of the center of the bathyphase), such that the position of the maximum or minimum may be determined more accurately. The sampling approach may significantly reduce computing complexity since the sensing activity may be significantly reduced. This may lead to a reduced energy consumption of the implantable device 100 which may increase the implantable device's longevity.

FIG. 3 shows a block diagram representing the functional relations of parts of an exemplary embodiment of the implantable device 100. For example, the implantable device 100 may comprise a temperature sensor unit 310, an analog-to-digital converter 320, a microcontroller 330 (alternatively also a microprocessor or any other processing unit may be provided), a timer 340. The outlined block diagram shows possible elements of the implantable device 100 when implanted into a blood vessel of a patient (e.g., in a pulmonary artery) and a corresponding mode of operation will be outlined below.

In general, the temperature sensor unit 310 may be used to determine/sense the temperature of the environment around the sensor unit 310. The temperature sensor unit 310 may comprise and/or be implemented by a temperature sensor for sensing/determining the temperature. The temperature sensor unit 310 may be based on any type of temperature sensor (e.g., an electrical temperature sensor, an integrated circuit sensor, a mechanical temperature sensor, etc.). In an example, the temperature sensor unit 310 may be based on a thermocouple, a resistive temperature device, a negative temperature coefficient thermistor, a resistance temperature detector, an infrared (temperature) sensor, a bimetallic device, a thermometer, a semiconductor-based (temperature) sensor. The temperature sensor unit 310 may be configured to determine a temperature (e.g., a body temperature) with an accuracy of at least 0.1° C.

For example, the temperature sensor unit 310 of the implantable device 100 may sense/acquire the temperature 300 of the surrounding environment when implanted into a patient. The temperature 300 may correspond to the body temperature of the patient having the implant. The temperature sensor unit 310 may convert the sensed temperature to an electrical signal which may correspond to the temperature 300. Subsequently, the electrical signal may be digitized into a digital signal by the analog-to-digital converter 320. The digital signal may be further processed by the microcontroller 330. For example, the microcontroller 330 may determine the temperature 300 of the environment based on the digital signal (e.g., match the digital signal with an absolute temperature and store the temperature as a variable and/or in a database).

In an example, the microcontroller may trigger acquiring the temperature (e.g., the body temperature BT) according to the concept laid out in FIG. 2 (i.e., by way of sampling the body temperature BT signal in predetermined time intervals). The microcontroller 330 may thus be configured to trigger discrete temperature senses and store the according temperature values (e.g., in a storage unit). In an example, the microcontroller 330 may be configured to derive a circadian rhythm, a circadian rhythm parameter, and/or a characteristic interval of the circadian rhythm based on the stored/acquired temperature values. The microcontroller 330 may execute various signal processing steps to the acquired temperature signal and/or acquired temperature values for said purpose (e.g., filtering, evaluating/determining signal characteristics (e.g., a mean value, effective value, a maximum value, a minimum value, creating statistics of the temperature, comparing with a threshold, feature extraction, pattern extraction, etc.)). Specifically, the microcontroller 330 may be configured to derive the bathyphase based on the stored temperature values. On the basis of the determined point of time (or the interval) of the bathyphase, the microcontroller 330 may start the timer 340. The timer 340 may activate the implantable device in regular intervals (e.g., daily, every 24 hours, every 48 hours, every 72 hours, etc.), wherein based on the activation the implantable device may determine the (blood) pressure (in other words, the timer may trigger a blood pressure measurement while at other times the pressure sensor is inactive). The regular intervals of the timer (first predetermined timing) may be integer multiples of 24 hours (e.g., n·24, wherein n=1, 2, 3, . . . , etc.) to match the circadian rhythm. Since the circadian cycle may repeat periodically every 24 hours this ensures that the blood pressure is determined in the same time window of a day the timer was initiated. With the timer being based on the bathyphase (i.e., phase of minimum physical activity), as outlined herein, it may be ensured that timer activated measurements of the blood pressure take place in the bathyphase. It may thus not be necessary to continuously determine the circadian rhythm and/or the bathyphase. However, in an example, the microcontroller 330 may be configured to check the point of time of the bathyphase in regular second intervals (according to a second predetermined timing). In an example, the microcontroller 330 may initiate deriving the point of time of the bathyphase every week, every month, every two months, etc. For example, the microcontroller may trigger acquiring the temperature (e.g., the body temperature BT) according to the concept laid out in FIG. 2 in the stated regular second intervals.

In another example, the microcontroller 330 may check in regular third intervals (that may be longer than the first intervals but shorter than the second intervals) the characteristics of the physiological parameter (e.g., the body temperature BT) at or in the vicinity of the (expected or extrapolated) bathyphase or any other characteristic interval of the circadian cycle. For example, the physiological parameter (e.g., temperature) may be acquired at the extrapolated time of tB (e.g., at tB+n*24 hours, wherein n is representative of the third interval) or around the extrapolated bathyphase (e.g., around tB+n*24 hours #m hours, m=0.5, 1, 2, 3, . . . ). For example, the microcontroller 330 may then determine, if the physiological parameter has undergone a significant change compared to the last known characteristics (e.g., if the temperature at tB+n*24 hours suddenly has a significantly higher value, if the minimum in the interval tB+3 hours is present at a significant offset from the center of this time interval, etc.). In an example, this approach may be considered a quick check if a subsequent deriving of the circadian cycle needs to be initiated.

In summary, the present invention reduces the support effort for patients having the implanted device 100 implanted for determining the blood pressure. At time changes and/or travels, no manual readjustment of the measurement time may be necessary. The measurement time may be automatically adapted to a patient specific day-night rhythm, which may facilitate reliable long-term pressure measurements.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Implantable device comprising:
means for deriving a circadian rhythm associated with a physiological parameter;
means for determining a blood pressure, based at least in part on the circadian rhythm.

2. Implantable device according to claim 1, further comprising means for acquiring the physiological parameter.

3. Implantable device according to claim 2, wherein the means for acquiring is configured to acquire the physiological parameter in predetermined time intervals.

4. Implantable device according to claim 1, wherein the means for deriving is configured to derive at least one circadian rhythm parameter based at least in part on the circadian rhythm.

5. Implantable device according to claim 4, wherein the means for determining is configured to determine the blood pressure based at least in part on the at least one circadian rhythm parameter.

6. Implantable device according to claim 4, wherein the at least one circadian rhythm parameter comprises a characteristic interval of the circadian rhythm.

7. Implantable device according to claim 6, wherein the characteristic interval comprises a bathyphase of the circadian rhythm and/or an acrophase of the circadian rhythm.

8. Implantable device according to claim 6, wherein the means for determining is configured to determine the blood pressure at least in part based on the characteristic interval.

9. Implantable device according to claim 8, wherein the means for determining is configured to determine the blood pressure based at least in part on a first predetermined timing relative to the characteristic interval.

10. Implantable device according to claim 1, wherein the means for deriving is configured to derive the circadian rhythm based at least in part on a second predetermined timing.

11. Implantable device according to claim 1, wherein the physiological parameter comprises at least one of the following: a body temperature, a heart rate, a respiratory rate, a sleeping pattern, a blood pressure, a hormone level, a melatonin level, a plasma level of cortisol, a body activity.

12. Implantable device according to claim 1, wherein the implantable device is configured for implanting into a blood vessel.

13. Implantable device according to claim 12, wherein the blood vessel is a pulmonary artery.

14. Method carried out by an implantable device comprising the following steps:
deriving a circadian rhythm associated with a physiological parameter;
determining a blood pressure, based at least in part on the circadian rhythm.

15. Computer program comprising instructions to perform the method of claim 14, when the instructions are executed by a processor.
